(19) United States
(12) Patent Application Publication
(10) Pub. No.: US 2006/0287364 A1
(43) Pub. Date: Dec. 21, 2006

(54) Methods of prevention of infection with HIV/AIDS, venereal disease and influenza

(76) Inventor: Yakov Yevseyevich Brilman, Voorhees, NJ (US)

Correspondence Address: FLASTER/GREENBERG P.C.;8 PENN CENTER, 1628 JOHN F. KENNEDY BLVD., 15TH FLOOR, PHILADELPHIA, PA, 19103, US

(21) Appl. No.: 11/454,444

(22) Filed: Jun. 15, 2006

Related U.S. Application Data
(63) Continuation-in-part of application No. 10/972,490, filed on Sep. 27, 2004.

(51) Int. Cl. A61K 31/4439; A61K 33/00; A61K 33/22

(52) U.S. Cl. 514/338; 424/660; 424/718

(57) ABSTRACT

The invention includes methods for prophylaxis of at least one of infection with HIV/AIDS and venereal diseases, grippe/influenza. The method includes use of materials, in solution, cream, gel and/or tablet form, which possess virucidal and bactericidal characteristics. The method includes applying such materials to a mammal's skin and/or mucous membranes in solution and/or cream or gel form. Also provided is a method applying such materials to the mouth and/or throat and nose for inhalation for prevention of and/or treatment of grippe and influenza. One such solution is an aqueous solution that includes about 0.1 to about 0.3 percent by weight pantoprazole or a derivative thereof, about 2 percent to about 4 percent by weight sodium chloride, about 0.1 percent to about 5 percent by weight sodium hydrocarbonate, and 0 percent to about 1 percent sodium tetraborate. Creams or gels including pantoprazole may also be used.

### CROSS-REFERENCE TO RELATED APPLICATIONS

This application is a continuation-in-part of U.S. patent application Ser. No. 10/972,490, filed Sep. 27, 2004, the entire disclosure of which is incorporated by reference herein.

### BACKGROUND OF THE INVENTION

AIDS is a severe or complete immunosuppressive disease that exposes patients to a wide range of infections and malignancies. In some cases, AIDS infection is accompanied by central nervous system disorders. Complete manifestation of AIDS is usually preceded by AIDS related complex (“ARC”), a syndrome accompanied by symptoms such as persistent generalized lymphadenopathy, fever and weight loss. Further, the human immunodeficiency virus (HIV) is associated as the etiological virus that leads to AIDS infection and its precursor, ARC.

Once a patient is infected with HIV virus, T4 lymphocytes become non-functional. As such T4 lymphocytes deplete, the AIDS/ARC disease increases. T cell depletion, and the ensuing immunological compromise, can be attributable to both recurrent cycles of infection and lytic growth and from cell-mediated spread of the virus.

One treatment for the prevention of the onset of HIV and AIDS is based upon soluble T4 protein. Proteins have been produced by recombinant techniques. Soluble T4 proteins interfere in a positive way with the T4/HIV interaction by blocking or competitive binding to inhibit HIV infection of cells expressing the T4 surface protein. Soluble T4 proteins are described as being useful as anti-viral therapeutics to inhibit HIV binding to T4+ cells and other virally induced effects.

Other proposed methods for treating or preventing AIDS have also focused on the development of anti-retroviral agents, which target the reverse transcriptase enzyme of HIV as a unique step in the life cycle of the virus. Examples of such agents include, for example, suramin, azidothymidine (“AZT”) and dideoxycytidine CH.

Although the above-noted prevention methods are promising, it is AZT that has demonstrated clinical benefits in clinical trials. An increasing number of patients receiving AZT, however, tolerate only low doses of the drug. Certain dosage regimens of AZT have been reported to be lymphotoxic. AZT administration in effective amounts has also been sometimes accompanied by undesirable and debilitating side effects.

Other prophylactic treatments use agents with anti-retroviral activity against steps in viral replication other than reverse transcription, including administering glucosidase inhibitors that ultimately contribute to a reduction in the expression of a functional envelope protein at the cell surface and the inhibition of production of infectious virus particles. Such anti-retroviral agents, however, have been described as potentially yielding toxic effects on cellular metabolism at higher doses in some cases.

Further, there are other methods of prevention including use of condoms and frequent testing to allow patients to seek early treatment. However, there still exists a need in the art for further alternative and/or effective treatments for the prevention of infection with HIV/AIDS (and ARC).

Grippe or influenza, otherwise known as influenza is difficult to prevent. In many cases, influenza shots or vaccines are recommended, but effectiveness can vary from patient to patient and virus strain to virus strain. This is due to the high mutability of the virus, so that generally an influenza vaccine formulation may only be good for about one year. The contents of the vaccine in any given year are formulated to contain the most likely strains to attack for the coming year.

People who receive the vaccination can still catch influenza. This is because of the mutability and because it is not possible to include all of the potentially different strains that can infect patients globally each season. It also takes time for manufacturers to formulate new vaccines and distribute the high numbers of dosages necessary to treat patients when there are influenza outbreaks. It is also possible become infected, get vaccinated soon thereafter before the onset of symptoms, and get sick from the strain the vaccine is formulated to prevent.

Vaccines can also cause the immune system to react as if the body is actually being infected resulting in the manifestation of symptoms even if such results are not as bad as actually catching a sustained influenza.

While an influenza vaccine is a relatively low risk vaccine, it can also still cause serious problems if there is a severe allergic reaction. As a result, in addition to vaccination, an individual's health level and maintaining adequate hygiene are important in avoiding and preventing influenza as well as to recovering therefrom.

In treating influenza, there are various methods including antiviral treatments, over-the-counter medicines such as decongestants and pain relievers, and more unconventional herbal treatments such as Echinacea for example. Rest, fluids and general over-the-counter medications are the most recommended forms of treatment.

Due to the difficulties in preventing influenza and the risk to human health, there is still a need in the art for additional and supportive methods of preventing and/or treating grippe/influenza.

PANTOCID (or PANTOSEPT) is known in tablet and injectible forms as a pharmaceutical preparation for treatment of gastrointestinal disorders, and also in tablet form for disinfection in water. It is also known for the irrigation and treatment of wounds in solution form (having 0.1 to 0.5% active ingredient).

Other components known in the art for various related uses, including hydrocarbonate sodium which is known for use in mouthwash or for treatment of head colds, conjunctivitis, laryngitis, stomatitis, and the like.

Further, it is known to incorporate sodium tetraborate for use in germicidal preparations, including use in mouthwash.

### BRIEF SUMMARY OF THE INVENTION

The invention includes a method for prophylaxis of the at least one of infection with HIV/AIDS virus, venereal disease, grippe and influenza, comprising externally treating a mammal with an effective amount of an aqueous solution comprising about 0.1 to about 0.3 percent by weight pantoprazole or a derivative thereof, about 2 percent to about 4 percent by weight sodium chloride, about 0.1 percent to about 5 percent by weight sodium hydrocarbonate, 0 percent to about 1 percent sodium tetraborate, based on the total weight of the solution; and externally applying an effective amount of a cream or gel based formulation comprising pantoprazole or a derivative thereof in an amount of about 0.1 to about 0.5 percent by weight, based on the total weight of the formulation.

Also included herein is a method for prophylaxis of infection with HIV/AIDS virus, comprising periodically externally treating a mammal by washing bowels of the mammal with an effective amount of an aqueous solution comprising about 0.1 to about 0.3 percent by weight pantoprazole or a derivative thereof, about 2 percent to about 4 percent by weight sodium chloride and about 0.1 percent to about 5 percent by weight sodium hydrocarbonate, based on a total weight of the aqueous solution or a second aqueous solution of 0.1 to about 0.5 weight percent pantoprazole based on a total weight of the second aqueous solution.

A method for prophylaxis of and/or treatment of grippe or influenza is also provided herein which method comprises applying to a mouth and/or throat and to a nose of a mammal an effective amount of an aqueous solution comprising about 0.1 to about 0.3 percent by weight pantoprazole or a derivative thereof, about 2 percent to about 4 percent by weight sodium chloride, about 0.1 percent to about 5 percent by weight sodium hydrocarbonate and 0 percent to about 1 percent sodium tetraborate, based on a total weight of the aqueous solution or a second aqueous solution of 0.1 to about 0.5 weight percent pantoprazole based on a total weight of the second aqueous solution.

### DETAILED DESCRIPTION OF THE INVENTION

The present invention includes materials for use in the prophylaxis of infection with HIV/AIDS virus as well as grippe and influenza. Such materials are available in solution form and/or as creams or gels and various other formulations. The materials can process through the skin and mucous membranes of a mammals body at locations where infections can typically enter after contact with a viral source or infected carrier of the associated virus or infection. As used herein, mammal includes both humans and animals, and the treatments may be directed to both and are preferably directed to treatment of humans.

A disinfecting effect is provided by solutions and creams or gels as described herein which contain active chlorine in safe concentrations and can quickly kill viruses associated with the above-noted infections.

A primary component in the materials of the invention is the pharmaceutical known as PANTOCID is commercially available in tablet and injectible form from Sun Pharmaceuticals Industries, Ltd., Mumbai. It is a pharmaceutical preparation based on pantoprazole and/or its derivatives such as pantoprazole sodium. Pantoprazole (PANTOCID or PANTOSEPT) is also known as halazone which is chemically identified as 4-(N,N-dichlorosulfamoyl)benzoic acid. Other derivatives/forms of this material include 4-[(dichloroamino)sulfonyl]benzoic acid, 4-[(dichloroamino)sulfonyl]benzoic acid, and the sodium salts thereof, as well as p-(dichlorosulfamoyl)benzoic acid, p-(dichlorosulfamoyl)-benzoic acid, and sodium salts thereof, dichlorosulfamidobenzoic acid, p-(N,N-dichlorosulfamyl)benzoic acid, p-(N,N-dichlorosulfamoyl)benzoic acid, p-carboxybenzenesulfondichloroamide, p-dichlorosulfamoylbenzoic acid, p-sulfondichloramidobenzoic acid, and parasulfondichloramido benzoic acid. This compound is also known and/or available as PANTOCID, PANTOCIDE, PANTOSEPT, PENTOCID and ZEPTABs.

As used herein, reference to pantoprazole is intended to encompass use of the basic compound 4-(N,N-dichlorosulfamoyl)benzoic acid as well as its derivatives including chlorinated and salt forms, variations, and unless expressly referred to otherwise, to encompass all available forms (tablets, injectible, solid, liquid solution form (diluted and concentrated) and the like).

The methods herein use various solutions, creams or gels in combinations. One such preferred solution is an aqueous solution of pantoprazole. In the aqueous solution, also known herein as “Brilman's Solution” or “Solution B”), pantoprazole (available as PANTOCID or more preferably as PANTOSEPT for washing) or a derivative thereof as noted herein is provided to the solution in amounts of about 0.1 to about 0.3 percent by weight, based on the total weight of the solution, along with about 2 to about 4 weight percent of sodium chloride, about 0.1 to about 5 percent by weight of sodium hydrocarbonate and optionally up to about 1 percent by weight sodium tetraborate. In one preferred solution, Solution B includes about 0.1 to about 0.2 percent by weight pantoprazole or a derivative thereof, about 4 percent by weight sodium chloride, about 5 percent by weight sodium hydrocarbonate, and about 0.5 percent by weight sodium tetraborate. The use of sodium hydrocarbonate (or hydrocarbonate sodium) assists in the dilution of the pantoprazole or its derivatives in the aqueous medium. This helps to increase the osmotic effect of the solution to promote improved cleansing of slime and other infected elements. Together, with a hypertonic solution of sodium chloride it increases the effect of abruption of the relevant virus from cell tissue. The alkaline nature of a preferred chorine-containing pantoprazole further assists in quick viral destruction.

The sodium tetraborate, if used in Solution B, further assists in the antiseptic effect of the solution.

It is preferred to use Solution B as a first treatment step in the prophylaxis of the various ailments noted above, by applying Solution B externally to the body in an effective amount. Such use provides partial disinfection. The solutions for external disinfection herein may be applied on the skin and mucous membranes in areas where infection can enter a mammal after contact with an infected and/or sick person or virus carrier. The solutions act by providing pantoprazole, preferably incorporating chlorine in safe concentrations, to quickly kill or disinfect the virus to which the user is exposed.

Solution B can also be used for cleaning of the skin and mucous membranes as well as infected areas of the body through use of Solution B in different forms, including as a mouthwash, washing solution, irrigation, spray and the like. The amount of Solution B needed will vary from patient to patient depending on use, and it will be understood that it can be added until an effective end result is provided.

It is preferred that after applying Solution B as a wash or the like externally to the body, a cream or gel based formulation is then applied to the area washed by Solution B. Such a cream or gel (or generally an unguent) may include as an active ingredient 0.1 to about 0.5 percent by weight, and preferably about 0.2 percent by weight of pantoprazole or a derivative thereof (preferably available as PANTOSEPT for this purpose). Such unguents are preferably applied to the skin and/or mucous membranes for about 15 to about 20 minutes, although it should be understood that such timing may vary from patient to patient. For prophylaxis of AIDS/HIV and venereal diseases, it is preferred that such unguents (and the washing with Solution B) be applied to the skin and/or mucous membranes of at least one of the sexual organs of the user, the anus, mouth and/or crotch area.

Solution B, prepared in one preferred embodiment uses pantoprazole (PANTOSEPT) in a mixture with sodium tetraborate and a hypertonic solution of sodium chloride and sodium hydrocarbonate. The mixture including the hypertonic solution of sodium chloride (preferably at about 4 weight percent) will promote the best cleansing of possible presence of virus on mucous membranes and skin exposed to infected material (bodily fluids, mucous, sperm and the like) due to its osmotic effects. Further, such preferred hypertonic salt solution also has germicidal characteristics.

In the method for prophylaxis of AIDS/HIV and/or venereal diseases, it is further preferred that in addition to the treatments noted above, for preventative purposes, periodic washing of the bowels and oral dosage administration of pantoprazole or its derivatives is also performed. In washing of the bowels, it is preferred to use a modified aqueous solution (Solution B-2) in which Solution B-2 is the same as Solution B but without the optional sodium tetraborate as a component. Oral dosage administration may be provided by tablet form (or other oral dosage forms—sprays, caplets, chewables or other available forms) of pantoprazole or its derivatives, preferably in the form of PANTOCID in 40 mg dosage tablet form. Such oral dosage is preferably administered only once per day for a period of time desired for prevention/prophylaxis.

As an alternative to the modified Solution B (Solution B-2), a second aqueous solution may be used which is simply a solution form of pantoprazole or a derivative thereof in an amount of about 0.1 to about 0.5 weight percent of pantoprazole or a derivative thereof as-an active agent in the second aqueous solution, and more preferably about 0.2 percent pantoprazole or a derivative thereof, based on the total weight of the second aqueous solution.

In prevention/prophylaxis of influenza (also known as grippe) or related viral infections and/or in treating such ailments, these solutions as noted above may also be employed. After contact with an infected mammal, the user may also externally apply the solutions to a patient, preferably by applying the solutions to the mouth and/or throat and also preferably to the nose of the exposed and/or infected person. Preferred for such prophylaxis or treatments are Solution B or the second aqueous solution described hereinabove. The solutions may be applied externally to the mouth using a mouthwash, to the throat, for example using a throat spray and/or to the nose using nose drops or nose spray. When using sprays, the exposed or infected person may inhale the solutions as well.

Following external application, the exposed or infected person may also apply the cream or gel noted above. Alternatively, the cream or gel, for the purpose of prophylaxis of influenza (gripped) may also include as an active ingredient about 0.1 to about 0.5 weight percent of oxolin based on the weight of the formulation, preferably about 0.2 percent oxolin (in combined form, as used herein, a cream or gel formulation including about 0.2 percent pantoprazole and 0.2 oxolin is noted herein as a cream or gel “oxopant”). Oxolin is a pharmaceutical active component typically available in ointment form for viricidal use. It is also known as oxolinium and can be available in that form and/or as 1,2,3,4-tetrahydro-1,2,3,4-tetraoxo-naphthalene dehydrate or in the form of oxolinic acid. As used herein oxolin includes this form as well as derivative forms useful for the same purpose and capable of carrying out the desired effects.

Further, when preparing the aqueous solution for external application to the mouth, throat and/or nose for use in an inhalable form, a solution “oxopant” can be prepared which is based on the second aqueous solution described above having about 0.1 to about 0.5 weight percent pantoprazole, preferably about 0.2 percent pantoprazole, which solution “oxopant” further includes about 0.3 to about 0.5 percent by weight oxolin.

In one preferred embodiment herein, a method for prophylaxis of infection with HIV/AIDS or venereal disease, Solution B having about 0.2% pantoprazole or a derivative thereof (preferably in the form of PANTOCID), about 4% sodium chloride, about 5% sodium hydrocarbonate and about 0.5 to about 1.0% by weight sodium tetraborate are combined in distilled water and applied externally to a person who may be exposed to the relevant virus.

Brilmans' Solution (Solution B)

| | |
|---|---|
| Pantocid ™ | 0.2% |
| Sodium Chloride | 4.0% |
| Sodium Hydrocarbonate | 5.0% |
| Sodium Tetraborate | 0.5% |
| Distilled Water | q.s. 100% |

MDS—External (for washing, mouthwashes, irrigation).

Solution B can be made by providing the active ingredients in the form of powder or tablets that can be dissolved in above noted formulations in water.

This Solution B and its variants as noted herein may be prescribed for broad use through pharmacies for various application methods including in solution form for irrigation, washing, inhalant spraying, and the like in the desired volumes.

Solution B and its variants as noted hereinabove may be thus used in the methods noted herein as a partial disinfection step and also for cleaning the skin and mucous membranes including those of the mouth, sexual organs, anus and crotch, as well as to remove any infected material through use of the solutions herein in the form of mouthwashes, for washing and for internal irrigation (such as washing of the vagina) after exposure (for example after sexual relations) to a possible virus carrier such as of HIV/AIDS or venereal disease.

After washing with this solution as noted hereinabove, it is preferred to further apply externally the unguent (preferably cream or gel) of pantoprazole or its derivatives (preferably in the form of PANTOSEPT) in an amount of about 0.1 to about 0.5 percent by weight, and preferably about 0.2 percent by weight. The unguent is put on the skin and mucous membranes as noted above preferably for about 15 to about 20 min.

It is preferred that the methods of application for prophylaxis noted herein be carried out as soon as is possible after sexual relations or other exposure for best results and to improve the ability to avoid contamination.

Considering that in early stages of contamination with HIV/AIDS infection there occurs a blocking of this virus in the bowels with mass destruction by the virus of “immune cells of the memory” (CD⁴+), it is preferred to also improve upon the washing and unguent application noted above by also using for preventive medical purposes an oral dosage of pantoprazole tablets, possessing virucidal characteristics. In preferred form, the oral dosage is a tablet having 40 mg. Such oral dosage should be taken once a day with water preferably for about 2 weeks or more, followed by or in conjunction with periodic washing of the bowels using the second aqueous solution noted hereinabove (preferably having from about 0.1 to about 0.2 weight percent pantoprazole in solution) or Solution B-2 (which as noted above is the same as Solution B, but exclusive of the optional sodium tetraborate).

The methods using the solutions herein can also be successfully used for the prophylaxis of influenza (grippe) and related viral infections and can be applied after contact with a sick/infected person by using the solutions for washing of the mouth and throat and/or dropping in the nose. Alternatively, the solutions may be applied by inhaling 2 to 3 sprays nasally and via the throat twice a day. For such prophylaxis, it is preferred to use Solution B. After such treatments with Solution B, it should be followed by application of the cream or gel noted herein to the mucous membranes, and more preferably use of the cream or gel noted above, and further including oxolin in the amounts noted above.

In addition, a solution as noted above, incorporating oxolin may be used for inhalation or spraying into the nose and throat about 2 times per day, mainly after contacting a sick or infected person or a visit to a public place where such infections are likely to be encountered (such as bus stations, buses, trains, train stations, stores, including contact with shopping cart handles, theatres, public bathrooms, kitchens and the like), particularly during an epidemic. Such solutions including oxolin may be prepared right before use in preferred embodiments to avoid break down or destruction of oxolin in the solution.

It should be noted that with respect to the various solutions, creams and ointments, if not contrary to the purposes intended herein, other active ingredients useful for prophylaxis of HIV/AIDS, venereal disease, influenza and the like may be provided in effective usage amounts to the solutions, creams, sprays and the like as noted herein. Further, standard inactive ingredients may be provided for thixotropic, pH, dissolution, storage, preservation, homogenization purposes, and the like, including colorants, fragrances, flavors, binders, preservatives, polymeric binders, stabilizers, oils, inactives, thickeners, and the like, provided such components do not otherwise substantially interfere with the active ingredients herein carrying out their effective purpose. Preferably, such additional inactives are provided in amounts useful for their standard purpose and further, it is preferred that such inactives be present in amounts not greater than about 50 to about 60 weight percent of the solution or other formulation.

Based on the foregoing, the present methods provide a prophylaxis for infection of various diseases and viral infections, which can be taken externally, including Solution B, Solution B-2, the unguent (cream or gel) pantoprazole formulation, and the second aqueous solution, whether used alone or as modified with oxolin. Further, methods are provided for treatments of various conditions as noted and which can be used as supplemental treatments in conjunction with known preventative treatments including vaccination, and other HIV/AIDS and venereal preventative treatments. Such methods as disclosed herein are also valuable in locations where access to other treatments or vaccines is not readily available. In contradistinction to vaccines, the proposed methods and solutions are able to act upon a variety of incidents of these diseases regardless of the variability of the virus or bacteria causing the disease. The methods and solutions are easily made and inexpensive without significant contraindications.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method for prophylaxis of the at least one of infection with HIV/AIDS virus, venereal disease, grippe and influenza, comprising
externally treating a mammal with an effective amount of an aqueous solution comprising about 0.1 to about 0.3 percent by weight pantoprazole or a derivative thereof, about 2 percent to about 4 percent by weight sodium chloride, about 0.1 percent to about 5 percent by weight sodium hydrocarbonate, 0 percent to about 1 percent sodium tetraborate, based on the total weight of the solution;
and externally applying an effective amount of a cream or gel based formulation comprising pantoprazole or a derivative thereof in an amount of about 0.1 to about 0.5 percent by weight, based on the total weight of the formulation.

2. The method according to claim 1, wherein the aqueous solution comprises 0.1 percent pantoprazole or a derivative thereof, 4 percent sodium chloride, 5 percent sodium hydrocarbonate, and 0.5 percent sodium tetraborate.

3. The method according to claim 1, wherein the cream or gel comprises about 0.2 percent by weight pantoprazole.

4. The method according to claim 1, wherein the method is for prophylaxis of infection with HIV/AIDS virus or venereal disease, and the method further comprises applying the aqueous solution to the mammal by at least one of washing an external surface of skin of the mammal and irrigating at least one mucous membrane, wherein the at least one mucous membrane is located on at least one of a sexual organ, anus, crotch and mouth.

5. The method according to claim 4, further comprising applying the cream or gel to at least one of the external surface of the skin of the mammal and the mucous membrane.

6. The method according to claim 1, wherein the method is for prophylaxis of infection with HIV/AIDS virus, the aqueous solution is free of the sodium tetraborate and the method further comprises applying the aqueous solution by periodically washing bowels of the mammal with the aqueous solution and wherein the cream or gel comprises 0.2 weight percent pantoprazole.

7. The method according to claim 1, wherein the method is for prophylaxis of infection of HIV/AIDS virus and the method further comprises administering to the mammal internally one dosage per day of about 40 mg pantoprazole.

8. The method according to claim 7, wherein the dosage is in tablet form.

9. The method according to claim 1, wherein the method is for prophylaxis of grippe and influenza and further comprises treatment of grippe and influenza, wherein the aqueous solution and then the cream or gel are applied to a mouth and/or a throat and a nose of the mammal.

10. The method according to claim 9, wherein the aqueous solution is applied as a mouthwash to the mouth, as a throat spray to the throat and as nose drops or a nose spray to the nose.

11. The method according to claim 9, wherein the cream or gel further comprises about 0.1 to about 0.5 percent by weight oxolin.

12. The method according to claim 11, wherein the cream or gel comprises about 0.2 percent by weight oxolin.

13. The method according to claim 1, wherein the method is for prophylaxis of grippe and influenza and further comprises treatment of grippe and influenza, wherein the method further comprises inhaling an aqueous solution comprising about 0.3 to about 0.5 percent by weight oxolin and 0.1 to about 0.5 percent by weight pantoprazole.

14. A method for prophylaxis of infection with HIV/AIDS virus, comprising periodically externally treating a mammal by washing bowels of the mammal with an effective amount of an aqueous solution comprising about 0.1 to about 0.3 percent by weight pantoprazole or a derivative thereof, about 2 percent to about 4 percent by weight sodium chloride and about 0.1 percent to about 5 percent by weight sodium hydrocarbonate, based on a total weight of the aqueous solution or a second aqueous solution of 0.1 to about 0.5 weight percent pantoprazole based on a total weight of the second aqueous solution.

15. The method according to claim 14, further comprises administering to the mammal internally one dosage per day of about 40 mg pantoprazole.

16. A method for prophylaxis of and/or treatment of grippe or influenza, comprising
applying to a mouth and/or throat and to a nose of a mammal an effective amount of an aqueous solution comprising about 0.1 to about 0.3 percent by weight pantoprazole or a derivative thereof, about 2 percent to about 4 percent by weight sodium chloride, about 0.1 percent to about 5 percent by weight sodium hydrocarbonate and 0 percent to about 1 percent sodium tetraborate, based on a total weight of the aqueous solution or a second aqueous solution of 0.1 to about 0.5 weight percent pantoprazole based on a total weight of the second aqueous solution.

17. The method according to claim 16, further comprising inhaling the second aqueous solution, wherein the second aqueous solution further comprises about 0.3 to about 0.5 percent by weight oxolin.

18. The method according to claim 16, wherein the aqueous solution is applied as a mouthwash to the mouth, as a throat spray to the throat and as nose drops or a nose spray to the nose.
